(19) United States
(12) Patent Application Publication
(10) Pub. No.: US 2014/0297300 A1
(43) Pub. Date: Oct. 2, 2014

(54) DISEASE MANAGEMENT SYSTEM AND METHOD USING A WIRED/WIRELESS COMMUNICATION NETWORK

(71) Applicants: Hong Jin Kim, Seoul (KR); Bong Jo Kwak, Seoul (KR); Geun Sik Kang, Seoul (KR)

(72) Inventors: Hong Jin Kim, Seoul (KR); Bong Jo Kwak, Seoul (KR); Geun Sik Kang, Seoul (KR)

(73) Assignee: INSUNG INFORMATION CO., LTD., Seoul (KR)

(21) Appl. No.: 14/113,345

(22) PCT Filed: Jun. 27, 2012
(86) PCT No.: PCT/KR2012/005067

§ 371 Date: Dec. 10, 2013

(30) Foreign Application Priority Data
Jun. 29, 2011 (KR) 10-2011-0064083

(51) Int. Cl. G06F 19/00; A61B 5/00

(52) U.S. Cl. CPC G06F 19/345; A61B 5/0022; A61B 5/742; A61B 5/7264; G06F 19/3418
USPC 705/2

(57) ABSTRACT

A disease management system and method that use a wired and wireless communication network, in which motivation and consistent encouragement to change a behavior of a user or a patient may be given by close acquaintances or other people who have knowledge about a corresponding disease, instead of an expert. Thus, with regard to disease management that consistently needs normal and frequent communication, a role of an expert may be minimized to a necessary degree, and a more effective and efficient health care service, compared to a conventional health care service, may be provided.

### CROSS-REFERENCE TO RELATED APPLICATIONS

This application is the National Stage of International Application No. PCT/KR2012/005067 International Filing date, 27 Jun. 2012, which designated the United States of America, and which International Application was published under PCT Article 21 (s) as WO Publication 2013/002546 A2 and which claims priority from, and the benefit of, Korean Application No. 10-2011-0064083 filed 29 Jun. 2011, the disclosures of which are incorporated herein by reference in their entireties.

### BACKGROUND

The presently disclosed embodiment relates to a disease management system and method using a wired and wireless communication network.

A health care service provides a convenient service to a user by integrating a medical service with information technology (IT). The health care service is one of the service models that are getting the spotlight in an aging society.

For example, when a user determines a physical condition of the user by using a measuring apparatus at home, a measurement result is transmitted to medical experts in real time. Thus, the health condition of the user may be consistently monitored by the medical experts. Additionally, a computer may automatically determine a physical condition of a user through urine or a pupil of the user, and transmit the determined physical condition to a medical center. Thus, the medical center may determine whether the physical condition of the user is normal, and guide the user towards a healthy lifestyle and a proper diet.

Additionally, when a patient checks a blood sugar level, blood pressure, body composition, and a stress level, a value that is obtained by the checking is automatically input to the measuring apparatus and transmitted to a doctor or a health manager. Thus, a physical condition of the patient is monitored in real time, and the patient may receive a health consultation through a video conference.

However, a typical health care service, described above, has an inherent limitation, since the conventional health care service is a unidirectional management model in which a user or a patient is managed based on unilateral instructions and supervisions that are made as determined by an expert, for example, a doctor, a nurse, a nutritionist, or a trainer. Since a patient is a passive subject in this model, in the case of disease management that aims to comprehensively change a patient's life style, there is a limit on motivating and encouraging the patient to voluntarily change his/her lifestyle. Additionally, since the conventional health care service is performed based on the expert's activities, comparatively, a lot of manpower and cost may be needed.

### SUMMARY

One or more aspects of the presently disclosed embodiment provides a disease management system and method that use a wired and wireless communication network and thus provide a service which is more effective and efficient than a conventional health care service. With regard to the disease management system and method, motivation and consistent encouragement to change a behavior of a user or a patient may be given by close acquaintances or other people who have knowledge about a disease, and thus, a role of an expert may be minimized to a necessary degree.

According to an aspect of the presently disclosed embodiment, there is provided a disease management method that uses a wired and wireless communication network, the disease management method including: collecting data regarding a patient's disease from a patient terminal; converting the collected data into a management index; sharing the management index with at least one other user terminal; receiving first feedback information regarding the management index from the at least one other user terminal; and sharing the first feedback information with the patient terminal.

The disease management method may further include sharing the management index with an expert server; receiving second feedback information regarding the management index from the expert server; and sharing the second feedback information with the patient terminal.

The disease management method may further include scoring the management index based on the first feedback information, and determining whether the scored management index is more than a first threshold; if the scored management index is more than the first threshold, generating first feedback data that is obtained by reflecting the first feedback information in the management index; and transmitting the generated first feedback data to the patient terminal.

The disease management method may further include, if the scored management index is less than the first threshold, sharing the management index with the expert server; receiving second feedback information regarding the management index from the expert server; generating second feedback data that is obtained by reflecting the second feedback information in the management index; and transmitting the generated second feedback data to the patient terminal.

The disease management method may further include displaying the first feedback data and/or the second feedback data.

The at least one other user terminal may be connected to a social network service (SNS) server, and thus, accesses the management index.

The patient terminal may include a mobile terminal of a patient or a biosensor of the patient.

According to an aspect of the presently disclosed embodiment, there is also provided a disease management system that uses a wired and wireless communication network, the disease management system including: a patient terminal; a disease management server that is connected to the patient terminal via the wired and wireless communication network; and a social network service (SNS) server that is connected to the disease management server, wherein the disease management server includes: a connection interface that manages a connection between the patient terminal and the SNS server; a data collection unit for collecting data regarding a patient's disease from the patient terminal; and a data management unit for converting the collected data into a management index, sharing the management index with at least one other user terminal, receiving first feedback information regarding the management index from the at least one other user terminal, and sharing the first feedback information with the patient terminal.

The disease management system may further include an expert server that is connected to the disease management server, wherein the data management unit shares the management index with the expert server, receives second feedback information regarding the management index from the expert server, and shares the second feedback information with the patient terminal.

The data management unit may score the management index based on the first feedback information, determine whether the scored management index is more than a first threshold, and if the scored management index is more than the first threshold, generate first feedback data that is obtained by reflecting the first feedback information in the management index, and the disease management system may further include a message transmission unit for transmitting the generated first feedback data to the patient terminal.

The disease management system may further include an expert server that is connected to the disease management server, wherein, if the scored management index is less than the first threshold, the data management unit shares the management index with the expert server, receives second feedback information regarding the management index from the expert server, and generates second feedback data that is obtained by reflecting the first feedback information in the management index, and the message transmission unit transmits the generated second feedback data to the patient terminal.

According to one or more of the above aspects of the presently disclosed embodiment, a disease management system and method that use a wired and wireless communication network allow to receive motivation and consistent encouragement to change a behavior of a user or a patient, from close acquaintances and other people. Additionally, with regard to disease management that consistently needs normal and frequent communication, a role of an expert may be minimized to a necessary degree. Thus, more effective and efficient health care service, compared to a conventional health care service, may be provided.

### BRIEF DESCRIPTION OF THE DRAWINGS

FIG. 1 is a schematic diagram of a disease management system according to an aspect of the presently disclosed embodiment;

FIG. 2 is a schematic diagram of the disease management server shown in FIG. 1 in detail, according to an aspect of the presently disclosed embodiment;

FIG. 3 is a schematic diagram of a data management unit shown in FIG. 2, according to an aspect of the presently disclosed embodiment;

FIG. 4 is a flowchart of a disease management method according to another aspect of the presently disclosed embodiment; and

FIG. 5 is a flowchart of a disease management method according to another aspect of the presently disclosed embodiment.

### DETAILED DESCRIPTION

Hereinafter, the presently disclosed embodiment will be described more fully with reference to the accompanying drawings, in which exemplary aspects of the presently disclosed embodiment are shown. In the description of the presently disclosed embodiment, certain detailed explanations of related art are omitted when it is deemed that they may unnecessarily obscure the essence of the invention.

The terminology used in the description of the specification and claims is for the purpose of describing particular aspects only and is not intended to be limiting of exemplary aspects. It will be further understood that terms, such as those defined in commonly used dictionaries, should be interpreted as having a meaning that is consistent with their meaning in the context of the relevant art.

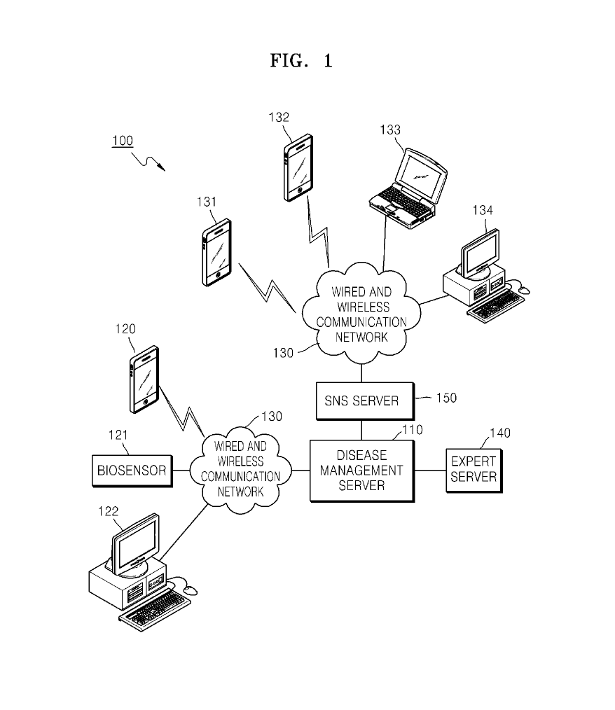

FIG. 1 is a schematic diagram of a disease management system 100 according to an aspect of the presently disclosed embodiment.

Referring to FIG. 1, the disease management system 100 includes a disease management server 110, patient terminals 120 through 122 that are connected via the disease management server 110 and a wired and wireless communication network 130, a social network service (SNS) server 150 and an expert server 140 which are connected to the disease management server 110, other user terminals 131 through 134 that are connected to the SNS server 150 via the wired and wireless communication network 130.

The patient terminals 120 through 122 may be a mobile terminal 120, a PC, a laptop computer 122, and a biosensor 121 that is attached to a body of a patient, respectively. The biosensor as the patient terminal 121 is a measuring apparatus that checks a blood sugar level, blood pressure, body composition, and a stress level, and then, automatically transmits a value, obtained by the checking, to the disease management server 110 via the wired and wireless communication network 130. Additionally, the patient terminals 120 through 122 automatically or manually transmit various information about patient activities, for example, exercise and nutrition, biometrics, for example, a vital sign such as blood pressure, a blood sugar level, weight/body fat, and cholesterol, and disease management activities of a patient, for example, medication and participation in health education to the disease management server 110 via the wired and wireless communication network 130.

The disease management server 100 collects data regarding a patient's disease from the patient terminals 120 through 122, and converts the collected data into a management index. The management index means an objective index on a state of a patient. A patient's behavior may be evaluated based on the management index. Additionally, the management index may protect a patient's privacy. The disease management server 110 shares a management index of a patient with the other user terminals 131 through 134 that are connected to the SNS server 150. The other user terminals 131 through 134 may be mobile terminals 131 and 132, a laptop computer 133, and a PC of a patient who has the same disease or a patient's guardian, respectively. For example, the disease management server 110 provides a community for patients, SNS acquaintances, a patients' club, or a guardian.

The disease management server 100 receives feedback information regarding a management index, which is shared with the other user terminals 131 through 134, from the other user terminals 131 through 134. The feedback information means information about opinions, encouragement messages, or motivation and consistent encouragement to change the patient's behavior, from patients with the same disease or other patients, with regard to a management index on the evaluation of a patient's behavior. For example, with regard to a management index of a patient who suffers from diabetes, for example, an eating habit, another patient who checked the eating habit may evaluate whether the eating habit is good or not, or give advice about food or an eating habit which is good for diabetes. The disease management server 110 shares feedback information, which is received from the other user terminals 131 through 134, with the patient terminals 120 through 122. Accordingly, a patient terminal may receive encouragement or motivation from the other user terminals 131 through 134. The disease management server 110 shares the management index with the expert server 140. Then, the disease management server 110 receives feedback information from the expert server 140, for example, an expert's opinion or a prescription on a patient's behavior, and shares the feedback information with the patient terminals 120 through 122. The expert server 140 may be a hospital server (not illustrated) or a server that serves a site for doctors, nurses, nutritionists, or trainers. Additionally, an expert, for example, a doctor, may provide feedback information about a management index via the expert's terminal, for example, a mobile terminal.

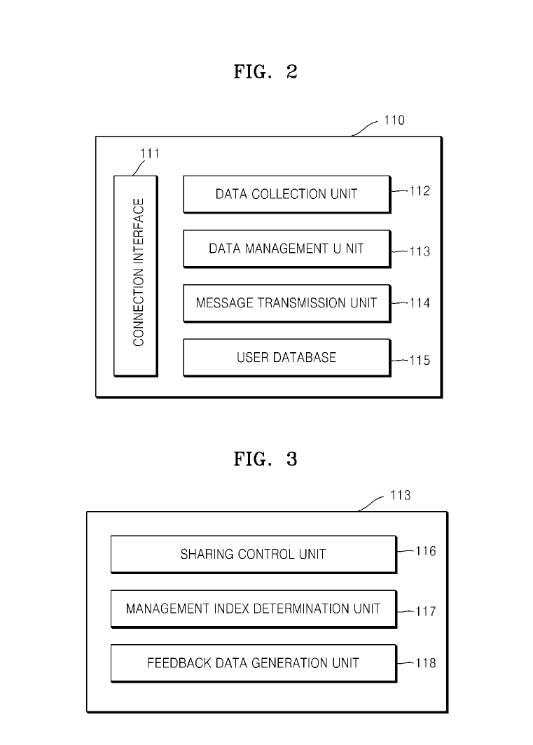

Additionally, the disease management server 110 scores a management index based on feedback information that is collected from the other user terminals 131 through 134. For example, by counting the number of advices or same advices on diabetes from the other user terminals 131 through 134, it is evaluated whether the advice on a patient's behavior is an appropriate evaluation or advice. The disease management server 110 determines whether the scored management index is more than a first threshold. If the scored management index is more than the first threshold, feedback data, which is obtained by reflecting feedback information in the management index, is generated and transmitted to the patient terminals 120 through 122. The first threshold is a value that may be arbitrarily set. Only when the scored management index, obtained by integrating the number of evaluations, agreements or disagreements which were made by the other user terminals 131 through 134 with regard to the management index, is a certain value or higher, the advice is given a reliability and provided as feedback to the patient. On the contrary, if the scored management index is less than the first threshold, the disease management server 100 shares the management index with the expert server 140, and reflects a feedback opinion of the expert. Then, the disease management server 100 transmits the feedback opinion of the expert to the patient terminals 120 through 122. The expert server 140 may be a hospital server (not illustrated), or a server that serves a site for doctors, nurses, nutritionists, or trainers. Referring to FIG. 1, the disease management system 100 is separated into the disease management server 110, the SNS server 150, and the expert server 140, and operated. However, the presently disclosed embodiment is not limited thereto, and the disease management server 110, in which the SNS server 150 and the expert server 140 are integrated, may be operated. A detailed configuration of the disease management server 110 will be described later by referring to FIGS. 2 and 3.

The SNS server 150 provides a service via which a person may have a social relationship with other people online. The SNS server 150 may be, for example, twitter, Wagle, me2day, Cyworld, or so on.

According to an aspect of the presently disclosed embodiment, the disease management system 100 is an SNS-based disease management system in which an SNS is applied to patient management, so as to change a patient's habit and behavior with regard to disease management, by motivating and monitoring interactions between patients. Additionally, a role of an expert in the SNS-based disease management system, for example, a role of a doctor, is to serve as a mentor or a consultant, and a main communication is made between a patient and relevant people, for example, a patient's guardian and an acquaintance. Through the communication, an active interaction for changing a patient's habit and behavior with regard to disease management may be made. In other words, motivation and consistent encouragement, which are necessary to change a patient's behavior, are provided through the SNS. Thus, with regard to disease management that consistently needs normal and frequent communication, a role of an expert may be minimized to a necessary degree.

FIG. 2 is a schematic diagram of the disease management server 110 shown in FIG. 1.

Referring to FIG. 2, the disease management server 110 includes a connection interface 111, a data collection unit 112, a data management unit 113, a message transmission unit 114, and a user database 115.

The connection interface 111 manages connection between the patient terminals 120 through 122, the SNS server 150, and the expert server 140.

The data collection unit 112 collects data regarding a patient's disease from the patient terminals 120 through 122.

The data management unit 113 manages collected data. A detailed configuration of the data management unit 113 is described with reference to FIG. 3.

Referring to FIG. 3, the data management unit 113 includes a sharing control unit 116, a management index determination unit 117, and a feedback data generation unit 118. The data management unit 113 converts collected data into a management index.

The sharing control unit 116 shares a management index with the other user terminals 131 through 134. Then, the sharing control unit 116 receives feedback information regarding the management index from the other user terminals 131 through 134 (the feedback information, received from the other user terminals 131 through 134, is defined as first feedback information). Additionally, the sharing control unit 116 shares the first feedback information with the patient terminals 120 through 122. Then, the sharing control unit 116 receives feedback information regarding the management index from the expert server 140, and shares the feedback, received from the expert server 140, with the patient terminals 120 through 122.

Additionally, if a scored management index is less than a first threshold, the sharing control unit 116 shares the scored management index with the expert server 140.

The management index determination unit 117 scores a management index based on the first feedback information, and determines whether the scored management index is more than the first threshold.

If the scored management index is more than the first threshold, the feedback data generation unit 118 generates first feedback data that is obtained by reflecting the first feedback information in the management index. Additionally, if the scored management index is less than the first threshold, second feedback data, in which feedback information that is received from the expert server 140 is reflected, is generated (the feedback information, received from the expert server 140, is defined as second feedback information).

The message transmission unit 114 transmits the first feedback data or the second feedback data, which is generated by the feedback data generation unit 118, to the patient terminals 120 through 122. The message transmission unit 114 may also transmit an emergency message to an emergency medical center based on the feedback data. For example, if it is determined that a patient's behavior is in an emergency or urgent situation according to a doctor's opinion, the message transmission unit 114 immediately transmits an emergency message to the emergency medical center.

The user database 115 databases information for managing, analyzing, and processing relevant health or medical information based on information or a management index regarding a patient.

Although not illustrated, the disease management system 100 may further include a system module that is necessary for executing functions for operating the disease management system 100, such as security and certification, so as to manage and control the whole disease management system 100.

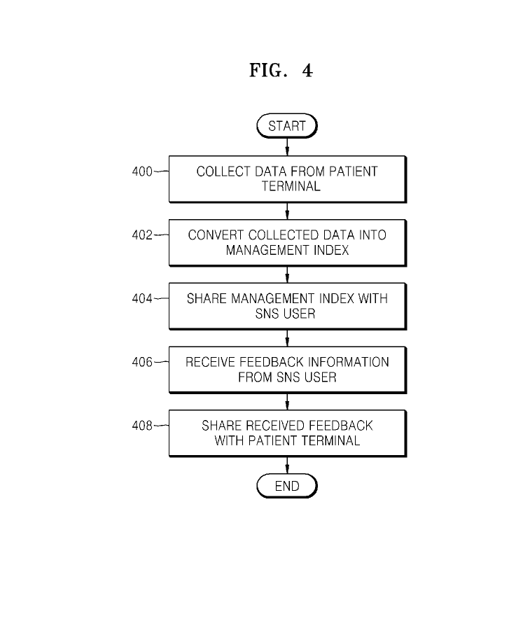

FIG. 4 is a flowchart of a disease management method according to another aspect of the presently disclosed embodiment.

Referring to FIG. 4, in operation 400, data is collected from the patient terminals 120 through 122. The data may include all information regarding a patient's behavior, for example, activity information that includes exercise and nutrition of the patient, biometrics that include blood pressure, a blood sugar level, weight/body fat, cholesterol, and so on, and disease management activities such as medication and participation in health education.

In operations 402 and 404, the collected data is converted into a management index, and the management index is shared with SNS users.

In operation 406, feedback information about the management index is received from the SNS users. The feedback information means information about opinions, encouragement messages, or motivation and consistent encouragement to change the patient's behavior, from patients with the same disease or other patients, with regard to a management index on the evaluation of a patient's behavior.

In operation 408, the received feedback information is shared with the patient terminals 120 through 122.

Although not shown in FIG. 4, sharing of the management index on the patient with the expert server, receiving feedback information regarding the management index from the expert server, and sharing the received feedback information with the patient terminals 120 through 122 may be further included.

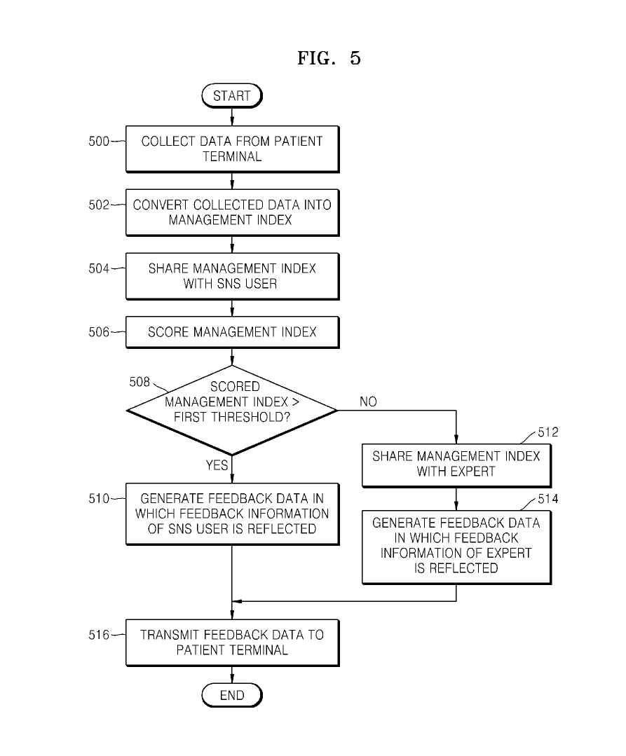

FIG. 5 is a flowchart of a disease management method according to another aspect of the presently disclosed embodiment.

Referring to FIG. 5, in operation 500, data is collected from the patient terminals 120 through 122. The data may include all information regarding a patient's behavior, for example, activity information that includes exercise and nutrition of the patient, biometrics that include blood pressure, a blood sugar level, weight/body fat, cholesterol, and so on, and disease management activities such as medication and participation in health education.

In operations 502 and 504, the collected data is converted into a management index, and the management index is shared with SNS users. In operation 506, the management index is scored. The scoring is performed in consideration of overall responses on the management index such as a degree of participation of SNS users, the number of replies, and agreements or disagreements.

In operation 508, whether the scored management index is more than a first threshold is determined. As a result of the determining in operation 508, if the scored management index is more than the first threshold, the method proceeds to operation 510, and generates feedback data in which feedback information of the SNS user is reflected.

Otherwise, in operation 508, if the scored management index is less than the first threshold, the method proceeds to operation 512, and shares the management index with an expert. Then, in operation 514, the disease management system 100 generates feedback data in which feedback information of the expert is reflected.

In operation 516, the feedback data is transmitted to the patient terminals 120 through 122.

Although not illustrated, prior to operation 518 or other operations, the generated feedback data may be displayed or disclosed on the disease management server 110, and notified to the patient or other patients.

According to an aspect of the presently disclosed embodiment, the disease management method using a wired and wireless communication network includes setting an objective management index on a state of a patient and evaluating the patient's behavior based on the management index, so as to primarily activate communication between a patient and relevant people instead of an expert. Additionally, the management index may be shared by SNS acquaintances, for example, a patient's club or a patient's guardian, so as to promote communication between the SNS acquaintances. Then, the management index may be converted into an appropriate score or point in consideration of a patient's privacy, and shared by the SNS acquaintances. Thus, the SNS acquaintances may actively give encouragement and advice, and participate in activities to change a behavior of a patient.

Additionally, if objective data, such as exercise data of a patient, can be collected through a sensor or a physical activity measuring system, active activities, encouragement, and joint participation may be promoted with regard to exercising by a patient, by increasing a degree of sharing the objective data between SNS acquaintances. Secondarily, in order to compensate for non-experts advice, an expert may support active communication between users by providing appropriate advice according to a change in the management index, and if necessary, participate in the management of a patient as an expert, through an active intervention, such as a remote health service.

The apparatus described herein may include a processor, a memory for storing program data and executing it, a permanent storage, such as a disk drive, a communications port for handling communication with external devices, and user interface devices, including a display, keys, etc. When software modules are involved, these software modules may be stored as program instructions or computer-readable codes executable on the processor on a computer-readable media such as read-only memory (ROM), random-access memory (RAM), CD-ROMs, magnetic tapes, floppy disks, and optical data storage devices. The computer-readable recording medium can also be distributed over network coupled computer systems so that the computer-readable code is stored and executed in a distributed fashion. This media can be read by the computer, stored in the memory, and executed by the processor.

All references, including publications, patent applications, and patents, cited herein are hereby incorporated by reference to the same extent as if each reference were individually and specifically indicated to be incorporated by reference and were set forth in its entirety herein.

For the purposes of promoting an understanding of the principles of the presently disclosed embodiment, reference has been made to the exemplary aspects illustrated in the drawings, and specific language has been used to describe these aspects. However, no limitation of the scope of the presently disclosed embodiment is intended by this specific language, and the presently disclosed embodiment should be construed to encompass all embodiments that would normally occur to one of ordinary skill in the art.

The presently disclosed embodiment may be described in terms of functional block components and various processing steps. Such functional blocks may be realized by any number of hardware and/or software components configured to perform the specified functions. For example, the presently disclosed embodiment may employ various integrated circuit components, e.g., memory elements, processing elements, logic elements, look-up tables, and the like, which may carry out a variety of functions under the control of one or more microprocessors or other control devices. Similarly, where the elements of the presently disclosed embodiment are implemented using software programming or software elements the invention may be implemented with any programming or scripting language, such as C, C++, Java, assembly language, or the like, with the various algorithms being implemented with any combination of data structures, objects, processes, routines or other programming elements. Functional aspects may be implemented in algorithms that execute on one or more processors. Furthermore, the presently disclosed embodiment could employ any number of conventional techniques for electronics configuration, signal processing and/or control, data processing and the like. The words “mechanism” and “element” are used broadly and are not limited to mechanical or physical embodiments, but can include software routines in conjunction with processors, etc.

The particular implementations shown and described herein are illustrative examples of the presently disclosed embodiment and are not intended to otherwise limit the scope of the invention in any way. For the sake of brevity, conventional electronics, control systems, software development and other functional aspects of the systems (and components of the individual operating components of the systems) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”.

The use of the terms “a” and “an” and “the” and similar referents in the context of describing the presently disclosed embodiment (especially in the context of the following claims) are to be construed to cover both the singular and the plural. Furthermore, recitation of ranges of values herein are merely intended to serve as a shorthand method of referring individually to each separate value falling within the range, unless otherwise indicated herein, and each separate value is incorporated into the specification as if it were individually recited herein. Finally, the steps of all methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the presently disclosed embodiment unless otherwise claimed. Additionally, it will be understood by those of ordinary skill in the art that various modifications, combinations, and changes can be formed according to design conditions and factors within the scope of the attached claims or the equivalents.

What is claimed is:

1. A disease management method that uses a wired and wireless communication network, the disease management method comprising:
collecting data regarding a patient's disease from a patient terminal;
converting the collected data into a management index;
sharing the management index with at least one other user terminal;
receiving first feedback information regarding the management index from the at least one other user terminal; and
sharing the first feedback information with the patient terminal.

2. The disease management method of claim 1, further comprising:
sharing the management index with an expert server;
receiving second feedback information regarding the management index from the expert server; and
sharing the second feedback information with the patient terminal.

3. The disease management method of claim 1, further comprising:
scoring the management index based on the first feedback information, and determining whether the scored management index is more than a first threshold;
if the scored management index is more than the first threshold, generating first feedback data that is obtained by reflecting the first feedback information in the management index; and
transmitting the generated first feedback data to the patient terminal.

4. The disease management method of claim 3, further comprising:
if the scored management index is less than the first threshold, sharing the management index with the expert server;
receiving second feedback information regarding the management index from the expert server;
generating second feedback data that is obtained by reflecting the second feedback information in the management index; and
transmitting the generated second feedback data to the patient terminal.

5. The disease management method of claim 4, further comprising displaying the first feedback data and/or the second feedback data.

6. The disease management method of claim 1, wherein the at least one other user terminal is connected to a social network service (SNS) server, and thus, accesses the management index.

7. The disease management method of claim 1, wherein the patient terminal comprises a mobile terminal of a patient or a biosensor of the patient.

8. A disease management system that uses a wired and wireless communication network, the disease management system comprising: a patient terminal; a disease management server that is connected to the patient terminal via the wired and wireless communication network; and a social network service (SNS) server that is connected to the disease management server,
wherein the disease management server comprises:
a connection interface that manages a connection between the patient terminal and the SNS server;
a data collection unit for collecting data regarding a patient's disease from the patient terminal; and
a data management unit for converting the collected data into a management index, sharing the management index with at least one other user terminal, receiving first feedback information regarding the management index from the at least one other user terminal, and sharing the first feedback information with the patient terminal.

9. The disease management system of claim 8, further comprising an expert server that is connected to the disease management server,
wherein the data management unit shares the management index with the expert server, receives second feedback information regarding the management index from the expert server, and shares the second feedback information with the patient terminal.

10. The disease management system of claim 8, wherein the data management unit scores the management index based on the first feedback information, determines whether the scored management index is more than a first threshold, and if the scored management index is more than the first threshold, generates first feedback data that is obtained by reflecting the first feedback information in the management index, and
the disease management system further comprises a message transmission unit for transmitting the generated first feedback data to the patient terminal.

11. The disease management system of claim 10, further comprising an expert server that is connected to the disease management server,
wherein, if the scored management index is less than the first threshold, the data management unit shares the management index with the expert server, receives second feedback information regarding the management index from the expert server, and generates second feedback data that is obtained by reflecting the first feedback information in the management index, and
the message transmission unit transmits the generated second feedback data to the patient terminal.

12. A non-transitory storage medium having stored thereon a computer program, which when executed by a computer, performs the method of claim 1.
